(19) United States
(12) Patent Application Publication
(10) Pub. No.: US 2011/0159057 A1
(43) Pub. Date: Jun. 30, 2011

(54) HYDROXYAPATITE AND BIOGLASS-BASED PELLETS, PRODUCTION PROCESS AND APPLICATIONS OF THEREOF

(76) Inventors: Jose Domingos Da Silva Santos, Porto (PT); Maria Ascensâo Ferreira Da Silva Lopes, Porto (PT); Marta Alves Da Silva, Ponte de Lima (PT)

(21) Appl. No.: 13/060,270

(22) PCT Filed: Aug. 22, 2008
(86) PCT No.: PCT/PT08/00032

§ 371 Date: Feb. 22, 2011

(51) Int. Cl. A61K 9/14; A61K 33/42; A61P 19/08; B29B 9/16; B29C 67/00

(52) U.S. Cl. 424/400; 424/602; 264/15

(57) ABSTRACT

The disclosed subject matter refers to hydroxyapatite and bioglass-based pellets of homogeneous size and spherical shape, whose interconnective porous structure, in the micrometer range, allows for an enhanced osteoconductivity and osteointegration, with specific application as a synthetic bone graft and to the respective production process. The production process is based on the pharmaceutical technology of extrusion and spheronization employing a porogenic agent and applying a sinterization stage in the presence of a vitreous liquid phase, which reverts on behalf of a higher reproducibility, superior yield and greater production capacity. Therefore, the disclosed subject matter is directed to the production of hydroxyapatite and bioglass-based pellets with applications in osteoregenerative medicine, particularly in the fields of orthopaedic surgery, maxillofacial surgery, dental surgery, implantology and as tissue engineering scaffolds

### FIELD OF THE INVENTION

The present invention refers to hydroxyapatite and bioglass-based pellets, their production process and respective applications, particularly as a synthetic bone graft. Such clinical applications are applied in all areas that include surgery and medicine, particularly those which are directly related with bone replacement and regeneration, such as orthopaedic surgery, maxillofacial surgery, dental surgery and implantology.

### BACKGROUND OF INVENTION

The bone is a complex mineralized tissue that exhibits rigidity and strength while maintaining a certain degree of elasticity, two forms existing, the primitive bone and lamellar bone. The first class is an immature bone that is formed during embryonic development, cicatrisation and fracture healing processes, tumours and metabolic diseases. Its structural organization is random. The lamellar bone is a more mature bone that gradually replaces the primitive bone, represents the major class of bone in the adult skeleton possessing a well organized structure. Namely, is constituted by cortical bone (external bone region) and trabecular bone (internal bone region). The cortical bone is characterized by cylindrical canals (osteons), united by a rigid tissue matrix which is essentially composed by hydroxyapatite. Collagen cylindrical fibres (the main organic component of bone) fill the pores (190-230 μm) of this kind of bone. The inorganic matrix of the cortical bone consists of a structure with approximately 65% interconnective porosity. On the other hand, the trabecular bone differs from the cortical bone by showing further empty spaces and non-cylindrical pores filled with collagen. Trabecular bone pores, in the range of 500-600 μm are larger than cortical bone pores. Therefore, it becomes apparent that due to its intrinsic complex structure, the bone is one of the most difficult tissues to mimic.

Currently, average life expectancy is twice as high as in the beginning of the 20^{th} century, resulting in a progressive tissue functionality loss. Of note, the incapacity associated to orthopaedic degeneration clinical challenges, which is considered a major social problem in modern society's aged populations. Actually, the bone is the second most transplanted material to the human body, only preceded by blood. Bone defects resulting from trauma, tumour resection, fracture non-union and congenital malformations are common clinical problems.

The consensual gold standard graft remains the autologous graft, consisting of bone collection in one site and transplantation to another site of the same individual. These grafts possess limitations concerning amount availability, as well as, the invasive nature of the harvest procedure. Due to their autologous origin, these grafts eliminate the risk of infection transmission (Human Immunodeficiency Virus, Hepatitis viruses, Creutzfeldt-Jakob disease) and/or of immunological rejection. However, high morbidity associated to donor site, as well as, local pain associated with the invasive harvest procedure extend the hospitalization period.

The alternatives to autologous grafts are allogenic grafts from post mortem human bone tissue and xenografts (non-human animal origin). Their clinical application introduces the possibility of immunological rejection, presents logistics problems and risk of infectious disease transmission to the recipient, which is currently a major concern of physicians, particularly in the case of viral diseases.

The use of synthetic bone grafts, namely, calcium phosphate ceramics, presents itself as the valid reference alternative due to its osteointegration ability. Hydroxyapatite, Ca₁₀(PO₄)₆(OH)₂, and tricalcium phosphate, Ca₃(PO₄)₂, comprise the most commonly used calcium phosphate ceramics in the clinical field owing to their similarity with bone mineral phase, and due to their biocompatibility, bioactivity and osteoconductivity properties.

Several studies attempted to obtain a production method of synthetic bone grafts with a micro and macroporous structure similar to the micro and macrostructure present in natural mineral bone (1-4). These studies focused their objectives in obtaining macrostructure, porosity, pore size, distribution and interconnectivity, which culminates in optimum osteoregeneration. Specifically, microporosity enhances cell adhesion and macroporosity foments bone growth within the bone graft, these factors being decisive for the increase in new bone growth rate locally at the implant site, as described below.

Attaining porosity in bone grafts has comprehended several methodologies, including foam and polymeric sponges-based technology and porogenic agents (1-4). In the first case, foams or polymeric sponges are impregnated with a biomaterial suspension and, upon drying, are processed by a thermal process which assures full combustion of the foam or sponge and concomitant formation of open pores (1, 2). The second technique employs different porogenic substances, such as organic additives and inorganic salts, which upon mixture with the ceramic biomaterial and subsequent appropriate thermal treatment, result in porous structures (3, 4).

However, these methods present recurring disadvantages that are due to non-controlled biomaterial retraction and residue presence after sintering, difficulty in controlling pore dimension, distribution and interconnectivity, and concomitant process reproducibility, presenting consequences at the level of cell colonization of the material. Additionally, elevated porosity percentages are associated to considerable mechanical resistance reduction compromising the clinical applications of the synthetic bone graft. On the other hand, in resorbable bone grafts, high porosity and consequent increase in specific surface area resulting in precocious resorption that might compromise bone regeneration due to the absence of physical support, as well as, to the induction of an inflammatory process. Therefore, a compromise between resorption rate and new bone growth rate becomes vital. In such compromise, and despite the reduction in mechanical resistance associated with the bone graft resorption rate, adequate percentages of micro and macroporosity will overpass those effects via bone cell and blood vessel ingrowth, which are the fundamental features for bone graft osteointegration.

Porosity characterized by pores with diameters equal to 100 μm is the fundamental condition for the capillary vascular growth and for the establishment of osteoprecursor cell-bone graft interactions which are essential for the growth and cell reorganization within the synthetic graft. Micro and macroporosity and pore interconnectivity degree, directly affect the diffusion of gas and nutrients present in physiological fluids, as well as, the metabolic residue removal. As cell growth occurs into the interior of the porous canals the bone graft acts as a structural bridge for bone regeneration.

Due to the abovementioned, the development of implantable biomaterials with porosity that mimics as much as possible the bimodal bone structure (cortical and trabecular) and that presents adequate interconnectivity degree, represents a tremendous challenge.

The present invention relates to a production process of hydroxyapatite and bioglass-based pellets (5), of homogeneous size and spherical shape, whose interconnective porous structure, in the micrometer range, allows for enhanced osteoconductivity and osteointegration. This kind of micro and macroporous structure is a fundamental requirement for the occurrence of cell adhesion and bone tissue growth within the material, which constitutes the first essential advantage of this novel biomaterial. The reproducibility of the pharmaceutical processes of extrusion and spheronization guaranties the above-mentioned characteristics, which in turn translates in a biomaterial whose behaviour is completely controlled and expected upon implantation. Additionally, the adaptation ability of spherical pellets to the form and geometry of the bone defect is extremely relevant, becoming also a fundamental advantage for the occurrence of enhanced osteoconduction and osteointegration.

The document WO 0068164 (5) discloses a material with applications as a bone graft, obtained through the reaction between a bioglass and hydroxyapatite, via a sintering process in the presence of a vitreous liquid phase that guaranties bioglass fusion and diffusion into hydroxyapatite structure which culminates in several ionic substitutions within its matrix. Such phenomenon confers the following characteristics to the bone graft: (a) Superior bioactivity, due to the reproduction of bone inorganic phase which contains several ionic species that modulate its biological behaviour, (b) Enhanced mechanical properties owing to the utilization of a bioglass of the CaO—P₂O₅ system that acts as liquid phase during the hydroxyapatite sinterization process and that, by filling the material pores, increases its density, and consequently, its mechanical resistance. Nevertheless, the bone graft production process described in the document WO 0068164 (5), does not result in a final product with a porous structure similar to the one of mineral bone, neither a macrostructure (or global geometry) considered ideal for clinical application in bone defects. The present invention discloses a production process of a bone graft comprising a bioglass, hydroxyapatite and at least one porogenic agent, through the pharmaceutical technology of extrusion and spheronization and a thermal process of sintering in the presence of a vitreous liquid phase. This process originates: (a) pellets, with spherical geometry considered ideal for the adaptation of the material to bone defects; (b) pellets with highly controlled micro and macroporous structures, which depends on the porogenic agent or porogenic agents used, and which is responsible for the osteoconduction and osteointegration of the bone graft.

Usually, market available synthetic bone grafts are produced in the form of granules obtained via a dry granulation process (U.S. Pat. No. 5,717,006 (6) and U.S. Pat. No. 5,064,436 (7)). Briefly, ceramic blocks, previously obtained by pressing and sinterization, are submitted to milling and size segregation. Despite the granules obtained accordingly to the mentioned method might present porosity, they exhibit irregular and angular geometry susceptible of inducing inflammatory reactions due to differences between individual granule reabsortpion rates and eventual tissue damage provoked by edges. Furthermore, the above-mentioned geometric irregularity makes the granules unsuitable for controlled drug release, due to the difficulty of a uniform coating with an active pharmaceutical substance. The biomaterial described in the present invention does not possess the previously mentioned disadvantages since it has a spherical form that is perfectly replicated via the extrusion and spheronization processes.

While US200406777001 (8) discloses a calcium phosphate ceramic sphere obtaining method consisting of the controlled dropping of the ceramic suspension into a low temperature medium, followed by a lyophilisation treatment of the frozen ceramic droplet and posterior sinterization, resulting in dense spheres, the production process disclosed in the present invention employs a pharmaceutical production process of extrusion and spheronization and a porogenic agent or agents for the production of hydroxyapatite and bioglass-based pellets (5), characterized by controlled aspect ratio and porosity, with diameters up to 10 mm. Moreover, and conversely to the process described in US200406777001 (8), the production process of the present invention is an automated, low cost and high productivity process, that during a short time span yields pellets of controlled aspect ratio and porosity, which allow for cellular adhesion and bone tissue ingrowth within the material.

While the process of pharmaceutical technology of extrusion and spheronization disclosed in EP1719503 (9) exclusively refers to the production of pellets with a formulation based on a debranched starch, several excipients and one or more active pharmaceutical agents, the production process disclosed in the present invention is based on the pharmaceutical technology of extrusion and spheronization using a porogenic agent or agents and hydroxyapatite sintering in the presence of a vitreous liquid phase in order to attain hydroxyapatite and bioglass-based ceramic pellets with controlled aspect ratio and porosity.

### GENERAL DESCRIPTION OF THE INVENTION

The present invention refers to hydroxyapatite and bioglass-based pellets, their production process and respective applications, particularly in osteoregenerative medicine as a bone graft.

The production process of these pellets is based in the pharmaceutical technology of extrusion and spheronization using a porogenic agent and a sintering process of hydroxyapatite in the presence of vitreous liquid phase, resulting in a low cost, high reproducibility, high yield and productive capacity. This process originates pellets with a granulometry superior to 10 mm, showing controlled porosity characterized by two pore populations. The pellets present homogeneous size and spherical shape, and an interconnective porous structure in the micrometer range.

### 1. Pellet Characteristics

The structures disclosed in the present invention are spherical-shaped, hydroxyapatite and bioglass-based, with a global porosity of at least 40 vol %, comprising an intraporosity (biomaterial pores) of at least 20 vol % and an interporosity (pores resulting from the biomaterial packing) of at least 20 vol %. The intraporosity, dependent on pellet size and on the porogenic agent used, is characterized by the presence of several distinct populations of pores: microporosity, with pores comprising diameters up to 5 μm; mesoporosity, with pores comprising diameters from 5-50 μm; macroporosity, with pores comprising diameters superior to 50 μm. The interporosity, dependent on pellet size, has pores comprising diameters superior to 10 μm.

### 2. Pellet Production Process

In the present invention, hydroxyapatite is prepared according to a precipitation method resulting from the reaction between a calcium hydroxide suspension (Ca(OH)₂) in purified water and an aqueous solution of orthophosphoric acid (H₃(PO₄)₂).

The bioglass employed in the production process of the present invention, belongs to the P₂O₅—CaO system, in a ratio of molar percentages of 20:80 to 80:20, with the possible nominal composition: CaF₂ (0-20 mol %), Na₂O (0-20 mol %) and MgO (0-20 mol %).

Bioglass preparation is performed via fusion of a sodium source (e.g., sodium carbonate (Na₂CO₃)), a calcium source (e.g., calcium hydrogenophosphate (CaHPO₄)), a fluor source (e.g., calcium fluoride (CaF₂), magnesium source (e.g., magnesium oxide (MgO)) and a phosphorus source (diphosphorus pentoxide(P₂O₅)).

Following the preparation of the abovementioned raw-materials, milling and sieving is performed in order to obtain particles with a granulometry up to 75 μm.

Afterwards, the biocompatible glass is added to hydroxyapatite in a weight percentage inferior to 10% relatively to the hydroxyapatite weight.

A porogenic agent, as disclosed in the present invention, is defined as any appropriate substance that makes the product suitable for extrusion and spheronization processes, having the ability to absorb and expand upon water retention and that upon sintering, suffers complete calcination not leaving any residue thus originating a porous structure. Preferably, the porogenic agent used ought to be at least one among cellulose, starch, modified starch, sorbitol, croscarmellose sodium, crospovidone, sodium alginate and lactose, among others, up to 80 wt % of the final mixture. The weight percentage at which the porogenic agent or agents are added is vital because besides accomplishing the desired porosity of the final biomaterial, it guaranties the desired plasticity of the initial paste, which is fundamental during the extrusion process. Paste plasticity is conferred through the hydration capacity of the porogenic agent or agents used, that upon mixture with hydroxyapatite and bioglass form an adequate plastic mixture for extrusion and spheronization, originating pellets of controlled aspect ratio and porosity.

The mixture procedure between hydroxyapatite, bioglass and porogenic agent or agents is performed via a dry process, employing a mixer, e.g., a double cone mixer, at a rate up to 100 rotations per minute (rpm) and during a period of time always superior to 5 minutes, in order to obtain a homogeneous powder blend that allows reproducibility of final product phase composition.

Subsequent to the powder dry mixture procedure, the granulation liquid, purified water, is gradually added at percentages between 50 wt % and 150 wt % relatively to powder mixture weight, depending on the porogenic agent or agents used and their respective water absorption capacity. The gradual addition is performed in a mixer, e.g., planetary mixer, in which the mixture is subsequently submitted to malaxation at a rate never inferior to 100 rpm for a period of time never inferior to 5 minutes, so as to attain a homogeneously lubrified paste. The moist paste obtained is then hydrated throughout a time period that can vary between 0.5 h and 36 h. These procedures have the purpose of granting appropriate rheologic properties, namely, plasticity and cohesion, which make the extrusion process of the mixture of hydroxyapatite, bioglass and porogenic agent or agents feasible.

After finalizing the hydration period, extrusion of the moist paste is performed using an extruder, e.g., roll extruder, provided with an extrusion screen up to 10 mm, at a rate inferior to 50 rpm. The extruder and screen type, as well as the extrusion rate greatly influence the extrudate characteristics. The roll extruder combines low pressure extrusion and low heat production with minimum water movement resulting in high product densification. The extrusion rate, the screen configuration and the extrusion temperature, significantly affect the water lubricant effect and the rheologic properties of the extrudate, consequently influencing the properties of the obtained pellets.

Next, the obtained extrudate is placed in a spheronizer that will never attain a rate inferior to 100 rpm, during a period of time never inferior to 1 minute. Spheronization rate is directly associated with the desired pellet size. Additionally, spheronization rate variations have a direct effect on the density, the hardness, spherical shape, porosity and superficial morphology of the pellets.

The attained pellets are dried in a forced air circulation oven, at a temperature never inferior to 60° C., until the water content in the pellets does not exceed 5 wt %. This drying procedure ensures the proper, structure non-damaging pellet manipulation before the sintering process.

Then, a thermal treatment of the pellets is performed, through temperature increase at a rate of 0.1-4° C./min, preferably at 0.5° C./min, until a temperature in the range of 400-800° C., preferably 600° C., is reached. The thermal treatment at the mentioned temperature takes place during a period of time not inferior to 1 h and 30 min in order to ensure the complete combustion of the porogenic agent or agents employed, without leaving residue while originating the porous structure.

Relatively to the sintering process, this should be performed above 1200° C., at a heating rate of 4° C./min, preferably at a temperature between 1250° C. and 1350° C., allowing the bioglass fusion and distribution in the hydroxyapatite matrix in a liquid phase sintering process. Once the sintering temperature is reached, the sintering thermal treatment in the presence of a vitreous liquid phase occurs during a period of time not inferior to 1 h, followed by the posterior natural cooling of the biomaterial to room temperature inside the furnace.

### 3. Advantages of the Pellet Production Process

The obtained structure of the hydroxyapatite and bioglass-based bone graft using the production process described in the present invention possesses several advantages.

The described process in the current invention presents low cost, high reproducibility, higher yield and productive capacity of the synthetic bone graft.

Concerning the reached porous structure, cell adhesion promotion and consequent cellular growth, namely, of osteoprecursor cells and blood vessels, induced by the release of ionic species from the biomaterial that culminates in a higher osteointegration and osteoregeneration are the main advantages. Furthermore, native conformation protein adsorption, present in physiological fluids, at the porous surface of the synthetic bone graft, contributes to an absent immunogenicity and a cellular proliferation increase.

The spherical shape of the pellets results in an adequate ability of injection and adaptation to any kind of bone defect. Therefore, the bone graft of the present invention could be used as an injectable composite material, consisting of the base biomaterial associated with a common biocompatible polymeric vehicle for minimal invasive surgery applications.

The homogenous size and spherical shape, and interconnective porosity of the pellets, further allow its application as a controlled pharmaceutical active substance release device, such as growth factors or other growth modulation and bone remodelling agents.

The synthetic bone graft pellets disclosed in the current invention have, therefore, several applications in osteoregenerative medicine, particularly in the fields of orthopaedic surgery, maxillofacial surgery, dental surgery, implantology and as tissue engineering scaffolds.

### DESCRIPTION OF THE DRAWINGS

FIGS. 1A and 1B: Pellets of 500-1000 μm granulometry, hydroxyapatite and bioglass-based, with controlled aspect ratio and porosity, prepared according to the method disclosed in the present invention, and observed by scanning electron microscopy (SEM).

FIG. 2: Granulometric distribution of hydroxyapatite and bioglass-based pellets with controlled aspect ratio and porosity, obtained with an extrusion screen of 1 mm, which reflects the reproducibility, higher yield and productive capacity of the method disclosed in the present invention.

FIG. 3: Pore distribution, mercury porosimetry-determined, of hydroxyapatite and bioglass-based pellets, obtained with an extrusion screen of 1 mm.

### DETAILED DESCRIPTION OF THE INVENTION

### 1. Pellet Production Process

The pellet production process of the present invention comprises hydroxyapatite and a bioglass of P₂O₅—CaO system preparation according to the following procedures:

### 1.1. Hydroxyapatite Preparation

Hydroxyapatite is prepared by precipitation of the product resulting of the reaction between a calcium hydroxide (Ca(OH)₂, >98%) suspension in purified water and an aqueous solution of orthophosphoric acid 85(wt/v) % (H₃(PO₄)₂) according to the following chemical reaction:

10Ca(OH)₂+6 H₃(PO)₄→Ca₁₀(PO₄)₆(OH)₂+18H₂O

After the preparation of the abovementioned raw material, milling and sieving are performed in order to obtain particles with a granulometry inferior to 75 μm.

### 1.2. Bioglass Preparation

The biocompatible glass with nominal composition [60-75%]P₂O₅-[0-25%]CaO-[0-15%]Na₂O-[0-15%]CaF₂-[0-20%]MgO (molar %) is prepared through a conventional melting process.

After the preparation of the abovementioned raw material, milling and sieving are performed in order to obtain particles with a granulometry inferior to 75 μm.

### 1.3. Raw Material Mixture

Afterwards, the bioglass is added to hydroxyapatite at a weight percentage inferior to 10% relatively to hydroxyapatite weight.

The addition of one or more porogenic agents to the hydroxyapatite and bioglass mixture is then performed, using at least, among others, cellulose, starch, modified starch, sorbitol, croscarmellose sodium, crospovidone, sodium alginate and lactose, up to 80 wt % of the final mixture.

The mixture procedure between hydroxyapatite, bioglass and porogenic agent or agents is performed via a dry process, employing a mixer, e.g., a double cone mixer, at a rate up to 100 rotations per minute (rpm) and during a period of time always superior to 5 minutes.

Subsequent to the powder dry mixture procedure, the granulation liquid, purified water, is gradually added at a percentage between 50 wt % and 150 wt % relatively to powder mix, depending on the porogenic agent or agents used and their respective water uptake. The gradual addition is performed in a mixer, e.g., planetary mixer, in which the mixture is subsquently, submitted to malaxation at a rate never inferior to 100 rpm during a period of time never inferior to 5 minutes.

The moist paste obtained is then hydrated throughout a time period that can vary between 0.5 h and 36 h.

### 1.4. Extrusion Process

Once the hydration period is complete, extrusion of the moist paste is performed using an extruder, e.g. roll extruder, provided with an extrusion screen up to 10 mm, at a rate inferior to 50 rpm.

### 1.5. Spheronization Process

The obtained extrudate is placed in a spheronizer that will never attain a rate inferior to 100 rpm, during a period of time never inferior to 1 minute.

### 1.6. Thermal Treatment

The attained pellets are dried in a forced air circulation oven, at a temperature never inferior to 60° C., until the water content in the pellets does not exceed 5 wt %.

Then, a thermal treatment of the pellets is performed, through temperature increase at a rate of 0.1-4° C./min, preferably at 0.5° C./min, until a temperature in the range of 400-800° C., preferably 600° C., is reached, during a period of time not inferior to 1 h and 30 min.

As far as the sintering process is concerned, this should be performed above 1200° C., at a heating rate of 4° C./min, preferably at a temperature between 1250° C. and 1350° C., using a liquid phase sintering process. Once the sintering temperature is reached, the sintering thermal treatment in the presence of a vitreous liquid phase occurs during a period of time not inferior to 1 h, followed by the subsequent natural cooling of the biomaterial to room temperature inside the furnace.

### 2. Pellet Characterization

The present invention discloses the production of synthetic hydroxyapatite and bioglass-based bone graft pellets, presenting a formulation up to 10 wt % of bioglass relatively to hydroxyapatite weight, and up to 80 wt % of at least a porogenic agent relatively to the hydroxyapatite and bioglass powder mixture weight.

The pellets disclosed in the present invention are characterized by a global porosity of at least 40 vol %, comprising an intraporosity (biomaterial pores) of at least 20 vol % and an interporosity (pores resulting from the biomaterial packing) of at least 20 vol %. The intraporosity, dependent on pellet size and on the porogenic agent used, is characterized by the presence of several distinct populations of pores: microporosity with pores comprising diameters up to 5 μm; mesoporosity with pores comprising diameters from 5-50 μm; macroporosity with pores comprising diameters superior to 50 μm. The interporosity, dependent on pellet size, is characterized in that it includes pores comprising diameters superior to 10 μm.

The present invention required granulometric distribution analysis through sieving, pore distribution analysis, porosity, surface area, average pore diameter, bulk and apparent density by means of mercury porosimetry. Pellet surface morphology was assessed by scanning electron microscopy (SEM). Additionally, resistance to crushing, the measurement of the necessary force to fracture the pellets, was performed. The pellet spherical degree was observed and calculated via aspect ratio (width/height) determination under an optical microscope. Such determination consists in calculating the ratio between the largest distance of a pellet (length) and the corresponding perpendicular dimension (height).

### EXAMPLES

### Example 1

### Hydroxyapatite, Bioglass-Based with at Least a Porogenic Agent Pellet Preparation with a Granulometry Between 500 to 1000 μm

### Hydroxyapatite Preparation

500.00 g hydroxyapatite are prepared by chemical precipitation according to the following chemical reaction:

10Ca(OH)₂+6H₃(PO)₄→Ca₁₀(PO₄)₆(OH)₂+18H₂O

In order to achieve that, 370.45 g calcium hydroxide (Ca(OH)₂, >98%), 345.15 g orthophosphoric acid 85 (wt/v) % (H₃PO₄) are weighed. 9 L purified water are poured in a large appropriated container, calcium hydroxide is added and mixed (Mixer R25) for 15 minutes. Meanwhile, 8 L purified water are poured in an appropriated recipient, orthophosphoric acid is added and the volume is completed with purified water up to 9 L. The addition of orthophosphoric acid is carried out via peristaltic pump (Minipuls 2) at a constant rate of 150 rpm. The mixture is performed for 4-5 hours, and cleaning of the calcium hydroxide container walls with purified water is required in order to prevent precipitate accumulation. Throughout the process, a pH control using a 32% ammonia solution is performed in order to maintain the pH higher than 10.5±0.5. After the acid solution addition, the container is washed with purified water and the rate of the peristaltic pump is increased to 360 rpm. Once the mixture is complete, the solution in the container is stirred for 1 hour followed by a resting period for of 16 hours where the mixture is left ageing. Afterwards, hydroxyapatite filtration is performed and dried in a forced air circulation oven (Binder). Once dried, hydroxyapatite is milled in a planetary mill (Fritsch Pulverizette 6) and sieved until a granulometry inferior to 75 μm is achieved.

### Bioglass Preparation

0.2 mol of a bioglass with the following nominal composition 65% P₂O₅-15% CaO-10% CaF₂-10% Na₂O (molar o) is prepared, wherein fluoride ion source is CaF₂. In order to achieve that, 2.12 g sodium carbonate (Na₂CO₃), 4.08 g calcium hydrogenophosphate (CaHPO₄), 1.56 g calcium fluoride (CaF₂) and 16.32 g diphosphorus pentoxide (P₂O₅) are weighed and mixed in a platinum crucible. The crucible is placed in a vertical furnace (Termolab) and heated for 1 h 30 min until 1450° C. are reached, followed by a dwelling time of 30 minutes, after which the molten glass is poured into purified water. Once the glass is dry, it is milled in a planetary mill (Fritsch Pulverizette 6) and sieved until a granulometry inferior to 75 μm is achieved.

### Pellet Preparation

487.50 g hydroxyapatite, 12.50 g bioglass and 500.00 g microcrystalline cellulose (Avicel PH101, with a diameter inferior to 50 μm) are mixed for 20 minutes at 150 rpm using a double cone mixer (ERWEKA). Then the mixture is placed on a planetary mixer (ERWEKA) and 825.00 mL purified water are gradually added for 5 minutes at 150 rpm. Afterwards, the paste malaxation procedure is performed, in the same ERWEKA planetary mixer at this instant provided with an adapter with planetary movement, for 10 minutes at 300 rpm. After the malaxation period, the moist paste is placed in a polyethylene air-deprived double bag, allowing the hydration of the microcrystalline cellulose for 2 h.

When the hydration period is complete, the moist paste is placed in a roll Caleva Screen Extruder 20, equipped with an extrusion screen with a 1 mm diameter, and at a rate of rpm the extrusion of the moist paste is performed. Following the extrusion process, the extrudate is placed in a spheronizer (Caleva Spheronizer 250), provided with a 3 mm spheronization plate, the rate is adjusted to 850 rpm and, after a 5 minute spheronization time, the pellets are removed.

The pellets are dried in a forced air circulation oven (Memmert), at a temperature never inferior to 60° C., until the water percentage in the pellets does not exceed 5 wt %, and a sintering thermal treatment of the pellets is then performed, at a heating rate of 0.5° C./min, up to 600° C. are reached and kept for a 90 minute period, followed by a heating rate of 4° C./min up to 1300° C. being this temperature maintained for 60 minutes, being followed by natural cooling inside the furnace. The first dwell time, performed at 600° C., is intended to attain complete combustion of the microcrystalline cellulose.

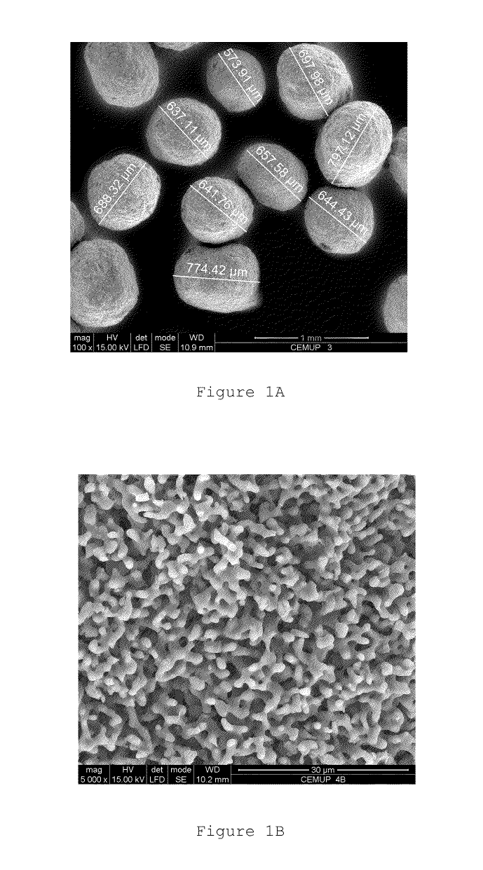

After the sintering, and relatively to the pellets morphology of the current example, these show an aspect ratio of 1.06 (FIG. 1A and Table 1), and their surface (FIG. 1B) is in agreement with the porosity revealed by the mercury porosimetry.

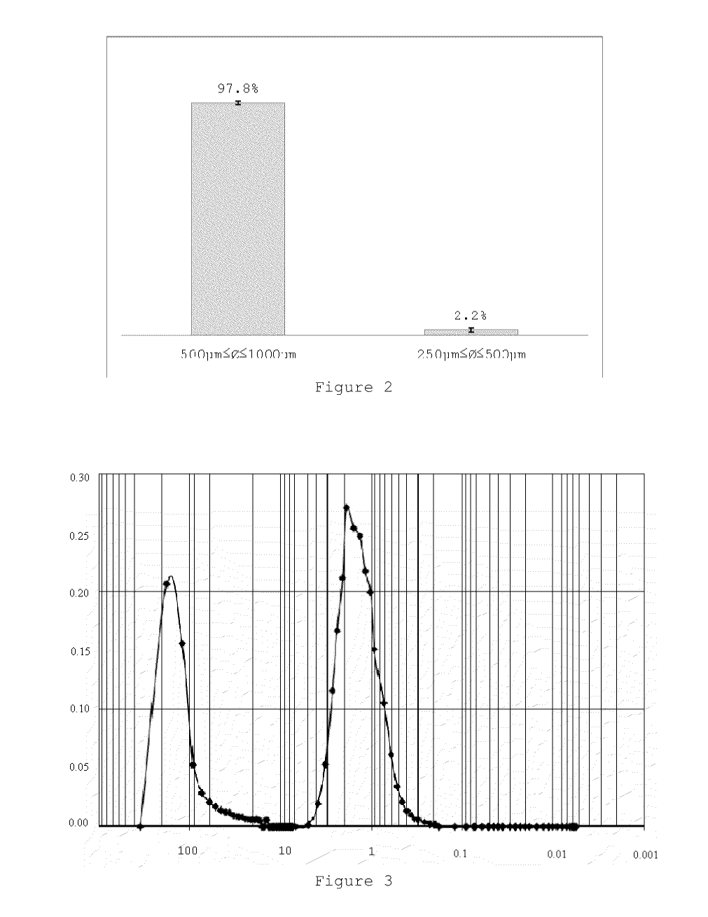

According to the present example, 97.8%±0.8% of the hydroxyapatite and bioglass-based pellets show a granulometry between 500 and 1000 μm (FIG. 2).

The pellets obtained according to the disclosed example, show a pore distribution depicted in FIG. 3, where it is possible to observe intra and interpores (the second and first peaks, respectively). The intraporosity obtained in the present example exhibits interconnective micro and mesopores (the second peak of FIG. 3).

TABLE 1
Characterization of hydroxyapatite and bioglass-
based pellets obtained by extrusion in a 1 mm
screen and spheronization process.

| | |
|---|---|
| Global Porosity (%) | 45.2 ± 4.4 |
| Intraporosity (%) | 24.6 ± 0.9 |
| Interporosity (%) | 20.6 ± 3.5 |
| Surface Area (m²/g) | 0.47 ± 0.04 |
| Bulk Density (g/mL) | 1.55 ± 0.20 |
| Apparent Density (g/mL) | 2.34 ± 0.02 |
| Crushing Resistance (N) | 5.2 ± 1.7 |
| Aspect ratio | 1.06 ± 0.05 |

Hydroxyapatite and bioglass-based pellet production process of the present example allows 45.2% global porosity resulting in a 0.47 m²/g surface area (Table 1). The attained intra and interporosities represent 24.6% and 20.6% in volume, respectively.

The attained pellets show a bulk density of 1.55 g/mL, an apparent density of 2.34 g/mL and a crushing resistance of 5.2; N (Table 1).

### BIBLIOGRAFIC REFERENCES

1) Lee L J, Zeng C, Cao X, Han X, Shen J, Xu G. Polymer nanocomposite foams. 2005. Composites Science and Technology, 65: 2344-2363.
2) Haugen P, Ried V, Brunner M, Will J, Wintermantel E. Water as foaming agent for open cell polyurethane structures. 2004. Journal of Materials Science: Materials in Medicine, 15: 343-346.
3) Prado da Silva M H, Lemos A F, Gibson I R, Ferreira J M, Santos J D. Porous glass reinforced hydroxyapatite materials produced with different organic additives. 2002. Journal of Non-Crystalline Solids, 304: 286-292.
4) Nam Y, Yoon J J, Park T. A Novel Fabrication Method of Macroporous Biodegradable Polymer Scaffolds Using Gas Foaming Salt as a Porogen Additive. 2000. J. Biomed Mater Res (Appl Biomater), 53: 1-7.
5) Santos J D, Hastings, G W, Knowles J C, Sintered hydroxyapatite compositions and method for the preparation thereof, WO 0068164, 2000.
6) Daculsi G., Weiss P., Delecrin J., Passuti N., Guerin F. Composition for biomaterial; Preparation process, U.S. Pat. No. 5,717,006, 1998.
7) Ogiso M., Ogawa T., Ichitsuka T., Inoue M. Bone prosthetic material, U.S. Pat. No. 5,064,436, 1991.
8) Umezu Y., Arai T. Method of production of ceramics, US200406777001, 2004.
9) Remon J. P., Dukic A., Altieri P. A., Vervaet C., Foreman P. B. Use of debranched starch in extrusion-spheronization pharmaceutical pellets, EP1719503, 2006.

1. Hydroxyapatite and bioglass-based pellets, wherein the pellets are not aggregated and they present a global porosity of at least 40 vol %, comprising an intraporosity of at least 20 vol % and an interporosity of at least 20 vol %.

2. Pellets, according to claim 1, wherein the bioglass employed in pellet production belongs to the P₂O₅—CaO system, in a ratio of molar percentages of 20:80 to 80:20, with the possible inclusion of CaF₂ (0-20 mol %), Na₂O (0-20 mol %) and MgO (0-20 mol %).

3. Pellets, according to claim 1, wherein the bioglass presents nominal composition [60-75%]P₂O₅-[0-25%]CaO-[0-15%]Na₂O-[0-15%]CaF₂-[0-20%]MgO (molar %).

4. Pellets according to claim 1, further comprising an intraporosity with several distinct populations of pores: microporosity, with pores comprising diameters up to 5 μm; mesoporosity, with pores comprising diameters from 5-50 μm; macroporosity, with pores comprising diameters superior to 50 μm.

5. Pellets according to claim 4, further comprising an interporosity with pores comprising diameters superior to 10 μm.

6. A process for producing the hydroxyapatite and bioglass-based pellets according to claim 1, wherein the process is carried out using the pharmaceutical technology of extrusion and spheronization employing at least one porogenic agent and a hydroxyapatite sintering process in the presence of a vitreous liquid phase, comprising the following steps:
a) mixing of hydroxyapatite with bioglass and at least one porogenic agent;
b) hydrating of the mixture resulting from the previous step;
c) extruding;
d) spheronization; and
e) thermal sintering treatment of resulting pellets.

7. The process according to claim 6, wherein at least one porogenic agent is used, being selected from a substance group such as cellulose, starch, modified starch, sorbitol, croscarmellose sodium, crospovidone, sodium alginate and lactose.

8. The process according to claim 6, wherein the pellet thermal treatment is initially carried out at a temperature within the range of 400-800° C.

9. The process according to claim 8, wherein the pellet thermal treatment is carried out at a temperature of 600° C.

10. Biomaterial, comprising the pellets recited in claim 1, and a common biocompatible polymeric carrier.

11. Biomaterial according to claim 10, wherein the biomaterial is used as a synthetic bone graft in surgery or human medicine related to bone substitution and regeneration, such as orthopaedic surgery, maxillofacial surgery, dental surgery and implantology.

12. Biomaterial according to claim 10, wherein the biomaterial is presented in injectable form.

13. Pellets, according to claim 2, wherein the bioglass presents nominal composition [60-75%]P₂O₅-[0-25%]CaO-[0-15%]Na₂O-[0-15%]CaF₂-[0-20%]MgO (molar %).

14. A process for producing the hydroxyapatite and bioglass-based pellets according to claim 2, wherein the process is carried out using the pharmaceutical technology of extrusion and spheronization employing at least one porogenic agent and a hydroxyapatite sintering process in the presence of a vitreous liquid phase, comprising the following steps:
a) mixing of hydroxyapatite with bioglass and at least one porogenic agent;
b) hydrating of the mixture resulting from the previous step;
c) extruding;
d) spheronization; and
e) thermal sintering treatment of resulting pellets.

15. A process for producing the hydroxyapatite and bioglass-based pellets according to claim 3, wherein the process is carried out using the pharmaceutical technology of extrusion and spheronization employing at least one porogenic agent and a hydroxyapatite sintering process in the presence of a vitreous liquid phase, comprising the following steps:
a) mixing of hydroxyapatite with bioglass and at least one porogenic agent;
b) hydrating of the mixture resulting from the previous step;
c) extruding;
d) spheronization; and
e) thermal sintering treatment of resulting pellets.

16. A process for producing the hydroxyapatite and bioglass-based pellets according to claim 4, wherein the process is carried out using the pharmaceutical technology of extrusion and spheronization employing at least one porogenic agent and a hydroxyapatite sintering process in the presence of a vitreous liquid phase, comprising the following steps:
a) mixing of hydroxyapatite with bioglass and at least one porogenic agent;
b) hydrating of the mixture resulting from the previous step;
c) extruding;
d) spheronization; and
e) thermal sintering treatment of resulting pellets.

17. A process for producing the hydroxyapatite and bioglass-based pellets according to claim 5, wherein the process is carried out using the pharmaceutical technology of extrusion and spheronization employing at least one porogenic agent and a hydroxyapatite sintering process in the presence of a vitreous liquid phase, comprising the following steps:
a) mixing of hydroxyapatite with bioglass and at least one porogenic agent;
b) hydrating of the mixture resulting from the previous step;
c) extruding;
d) spheronization; and
e) thermal sintering treatment of resulting pellets.

18. Biomaterial, comprising the pellets recited in claim 2, and a common biocompatible polymeric carrier.

19. Biomaterial, comprising the pellets recited in claim 3, and a common biocompatible polymeric carrier.

20. Biomaterial, comprising the pellets recited in claim 4, and a common biocompatible polymeric carrier.
